(19) United States
(12) Patent Application Publication
(10) Pub. No.: US 2026/0207289 A1
(43) Pub. Date: Jul. 23, 2026

(54) SYSTEM AND METHOD FOR MULTI-CHANNEL FLUORESCENCE SURGICAL MICROSCOPY

(71) Applicant: Ramona Optics Inc., Durham, NC (US)

(72) Inventors: Mark Harfouche, Durham, NC (US); Gregor Horstmeyer, Durham, NC (US); Aurélien Gauthier Florent Bègue, Durham, NC (US); Jed Doman, Durham, NC (US); Paul Reamey, Durham, NC (US)

(73) Assignee: Ramona Optics Inc., Durham, NC (US)

(21) Appl. No.: 19/455,338

(22) Filed: Jan. 21, 2026

Related U.S. Application Data
(60) Provisional application No. 63/748,155, filed on Jan. 22, 2025.

(51) Int. Cl. A61B 90/20

(52) U.S. Cl. CPC A61B 90/20

(57) ABSTRACT

A system and method for multi-channel fluorescence surgical microscopy are disclosed. A compact fluorescence multi-camera array includes more than two micro-cameras positioned behind a primary objective lens, with subsets of the micro-cameras configured for bright-field imaging and for different fluorescence emission channels. The system simultaneously captures multiple fluorescence channels together with multi-perspective image data, which are processed to generate quantitative three-dimensional information at video rates and high spatial resolution. An illumination system provides concurrent bright-field illumination and multi-channel fluorescence excitation. The resulting three-dimensional and multi-spectral information is displayed in real time and may be used to guide surgical visualization, navigation, or robotic-assisted procedures.

### CROSS-REFERENCE TO RELATED APPLICATIONS

This application claims the benefit under 35 U.S.C. § 119(e) of U.S. Provisional Patent Application No. 63/748,155, filed on January 21, 2025, entitled “SYSTEM AND METHOD FOR MULTI-CHANNEL FLUORESCENCE SURGICAL MICROSCOPY,” the entire contents of which are incorporated herein by reference.

### FIELD OF THE INVENTION

The present disclosure relates generally to microscopy and imaging technology and particularly, to a system and method to achieve multi-channel fluorescence imaging of more than two channels, with the ability to measure quantitative 3D information, in real time at high resolution for visual observation during surgical settings.

### BACKGROUND OF THE INVENTION

Surgical microscopes are routinely used within the general field of microsurgery to observe surgical areas at resolutions higher than the naked eye. Such microscopes are also commonly used in biomedical research to observe model organisms like the mouse, rat, and non-human primate. Increasingly, such microscopes, which typically image over centimeter-scale areas at anywhere between 1-100 µm optical resolution, are increasingly designed as digital microscopes. Companies like Zeiss, Leica and Synaptive Surgical all make such digital microscopes, also termed exoscopes. Some exoscopes contain two cameras that directly image the surgical surface at requisite resolutions for stereoscopic capture of two perspectives, which are then directly shown on two image channels of a stereoscopic display.

However, no existing digital surgical microscopes provide direct and quantitative measurements of the height of 3D surfaces that are independent of illumination. Certain high-resolution microscopic imaging modalities can offer quantitative 3D imaging capabilities. Examples include 3D imaging via the use of time-of-flight capture, structured illumination, holography, and optical coherence tomography. However, all such techniques depend upon an external beam of light and are not directly compatible with fluorescence imaging. Additional examples that are not dependent on active illumination or interference include confocal scanning methods, which are useful at very high resolution but are not suitable for larger-scale imaging in surgical scenarios nor for real-time synchronized imaging for live viewing.

In addition, some current exoscopes can capture fluorescence images and video for fluorescence guided surgeries. However, all such existing methods known to date just capture one or two fluorescence channels at most, and cannot simultaneously capture more than two data channels for simultaneous display.

Further, the use of multi-color illumination for surgical imaging has been previously described, but this approach did not include simultaneous multi-spectral capture nor 3D imaging capabilities (See, e.g., US Patent No. 12,004,722).

Moreover, light field microscopes and recent Fourier light field setups can uniquely capture 3D video at high speeds, but existing light field formats must directly tradeoff spatial resolution for the ability to acquire 3D information, which precludes high-resolution, large-area capture (Levoy, Zhang, & McDowell (2009). “Recording and controlling the 4D light field in a microscope using microlens arrays,” J. Microscopy 235(2):144-162; Broxton et al. (2013). “Wave optics theory and 3-D deconvolution for the light field microscope,” Opt. Express 21:25418-25439; Guo et al. (2019). “Fourier light-field microscopy,” Opt. Express 27:25573-25594 (2019)).

Nearly all current fluorescence microscopes support just a single color in 2D. The present invention provides a 3D multi-channel fluorescence operation that can directly enhance procedures where joint visualization of vasculature, tumor and healthy tissue is critical as well as operations where quantitative 3D measurements can better guide stereotactic and robotic equipment, for example.

Multiple recent studies have used dual-wavelength fluorescence microscopy (i.e., acquiring two spectral channels) for surgical guidance (van Beurden et al., “Multi-wavelength Fluorescence in image-guided surgery, clinical feasibility and future perspectives,” Mol. Imaging 19:153601212096233 (2020); van Willigen et al., “Multispectral fluorescence guided surgery; a feasibility study in a phantom using a clinical-grade laparoscopic camera system,” Am. J. Nucl. Med. Mol. Imaging 7(3):138–147 (2017); Acerbi et al., “Feasibility of simultaneous sodium fluorescein and indocyanine green injection in neurosurgical procedures,” Clin. Neurol. Neurosurg. 146:123–129 (2016); Yano et al., “Pathological analysis of the surgical margins of resected glioblastomas excised using photodynamic visualization with both 5-aminolevulinic acid and fluorescein sodium,” J. Neurooncol. 133(2):389–397 (2017); Suero Molina et al., “Dual-labeling with 5-aminolevulinic acid and fluorescein for fluorescence-guided resection of high-grade gliomas: technical note,” J. Neurosurg. 128(2):399–405 (2018)). The brand new da Vinci® FGS endoscope can now jointly record Fluorescein and indocyanine green (ICG) (Meershoek et al., “Multi-wavelength fluorescence imaging with a da Vinci Firefly—a technical look behind the scenes,” J. Robotic Surg. 15, 751–760 (2021)). Benefits of imaging two fluorescence dyes during surgery include supporting visualization of separate surgical targets (e.g., vasculature, malignant tissue, nerves), differentiating surgical targets from vital structures, and detecting unique molecular targets for precision surgery (van Beurden et al., “Multi-wavelength Fluorescence in image-guided surgery, clinical feasibility and future perspectives,” Mol. Imaging 19:153601212096233 (2020)). Applications include glioma imaging (Maugeri et al., “With a little help from my friends: the role of intraoperative fluorescent dyes in the surgical management of high-grade gliomas,” Brain Sci. 8(2):31 (2018); Acerbi et al. “Feasibility of simultaneous sodium fluorescein and indocyanine green injection in neurosurgical procedure,” Clin. Neurol. Neurosurg. 146:123–129 (2016)), angiographic imaging of cerebral aneurysms (Lane et al., “A prospective com- parative study of microscope-integrated intraoperative fluorescein and indocyanine videoangiography for clip ligation of complex cerebral aneurysms.” J. Neurosurg. 2015;122(3): 618–626), and staining glioblastoma (Della Puppa et al., “Combined fluorescence using 5-aminolevulinic acid and fluorescein sodium at glioblastoma border: intraoperative findings and histopathologic data about 3 newly diagnosed consecutive cases,” World Neurosurg. 122:e856–e863 (2019)) and the brain stem (Suero Molina & Stummer, “Where and when to cut? Fluorescein guidance for brain stem and spinal cord tumor surgery- technical note,” Oper. Neurosurg. 15(3): 325–331 (2018)). The present invention extends such exciting efforts to jointly capture 4 spectral channels in quantitative 3D, where >4 channels is directly possible in future efforts.

Related work has also implemented hyperspectral imaging of many spectral channels during surgery (Kotwal et al., “Hyperspectral imaging in neurosurgery: a review of systems, computational methods, and clinical applications,” J. Biomed. Opt. 30(2) 023512 (2024)). Most of these systems detect scattered bright-field light (i.e., not fluorescence) and measured each spectral channel sequentially over time (van Willigen et al., “Multispectral fluorescence guided surgery; a feasibility study in a phantom using a clinical-grade laparoscopic camera system,” Am. J. Nucl. Med. Mol. Imaging 7(3):138–147 (2017)), which unfortunately requires a static surgical area and cannot support real-time interactive viewing. Several “snapshot” multispectral imaging methods have been developed to support real-time viewing (Hagen & Kudenov, “Review of snapshot spectral imaging technologies,” Opt. Eng. 52(9):090901 (2013); Pichette et al., “Intraoperative video-rate hemodynamic response assessment in human cortex using snapshot hyperspectral optical imaging,” Neurophotonics 3(4): 045003 (2016); MacCormac et al., “Lightfield hyperspectral imaging in neuro-oncology surgery: an IDEAL 0 and 1 study,” Frontiers in Neuroscience 17:1239764 (2023); Vandebriel et al., “Integrating hyperspectral imaging in an existing intra-operative environment for detection of intrinsic brain tumors,” Proc. SPIE 12368, 123680D (2023) ; Saragadam et al., “SASSI—super-pixelated adaptive spatio-spectral imaging,” IEEE Trans. Pattern Anal. Mach. Intell. 43(7), 2233–2244 (2021)), but all significantly reduced spatial resolution for spectral capture (typically by >25X) and thus found limited practical use (Kotwal et al., “Hyperspectral imaging in neurosurgery: a review of systems, computational methods, and clinical applications,” J. Biomed. Opt. 30(2) 023512 (2024)). The present invention overcomes such resolution tradeoffs by developing a compact multi-camera design that simultaneously captures 4 spectral channels with over 4K resolution each, and with quantitative 3D. See, for example, U.S. Patent 11,857,317.

In short, there currently are no video-rate multi-channel fluorescence microscopes that fit within a compact form factor for dynamic positioning over the curved body, where 3D capture is critical or effective observation and operation. Accordingly, there is an urgent need to overcome the various limitations of available systems with respect to multi-channel fluorescence imaging of more than two channels, with the ability to measure quantitative 3D information, in real time at high resolution for visual observation during surgical settings.

### SUMMARY OF THE INVENTION

The present disclosure provides a system and method for multi-channel fluorescence surgical microscopy capable of simultaneously acquiring more than two fluorescence channels together with quantitative three-dimensional (3D) spatial information at video rates and high spatial resolution. The system, referred to herein as a fluorescence multi-camera array (FMCA), overcomes limitations of existing surgical microscopes and exoscopes that are restricted to one or two fluorescence channels and lack quantitative 3D measurement capability.

In one aspect, the invention comprises a compact array of more than two micro-cameras positioned behind a primary objective lens. Each micro-camera includes an image sensor and an associated imaging optic, and the micro-cameras are arranged to capture radiation from a surgical field from multiple angular perspectives. A subset of the micro-cameras is configured to capture bright-field imagery, while other subsets are provided with distinct spectral emission filters corresponding to different fluorescence channels. The filtered micro-cameras simultaneously capture fluorescence emission from multiple fluorophores without temporal multiplexing.

In another aspect, the FMCA includes an illumination system configured to simultaneously provide bright-field illumination and multi-channel fluorescence excitation. The illumination system may include one or more external ring light sources, internal light sources, or combinations thereof, with independently controllable excitation wavelengths and intensities corresponding to the fluorescence channels.

In a further aspect, synchronized image data from the micro-camera array are processed by one or more processors to generate quantitative 3D information using multi-view geometry while preserving high spatial resolution. The system further processes the multi-channel fluorescence data to generate registered multi-spectral representations of the surgical field. The resulting 3D and multi-channel fluorescence information may be displayed in real time, overlaid, selectively toggled, or used to guide surgical instruments, robotic systems, or other peripheral devices.

Advantageously, the disclosed system enables real-time, high-resolution, quantitative 3D visualization of multiple fluorescence markers during surgery within a compact form factor suitable for dynamic positioning over a patient. The invention thereby enhances fluorescence-guided surgery, stereotactic navigation, and robotic-assisted procedures.

The foregoing paragraphs have been provided by way of general introduction and are not intended to limit the scope of the following claims. The described embodiments, together with further advantages, will be best understood by reference to the following detailed description taken in conjunction with the accompanying drawings.

### BRIEF DESCRIPTION OF DRAWINGS

Having thus described the subject matter of the present invention in general terms, reference will now be made to the accompanying drawings, which are not necessarily drawn to scale, and wherein:

FIG. 1 illustrates a schematic illustration of the fluorescence multi-camera array microscope imaging a surgical area and displaying the resulting data on a nearby display;

FIG. 2 illustrates a schematic illustration of the system arranged to acquire multiple angular perspectives and multiple filtered fluorescence channels simultaneously, for Quantitative 3D imaging and multi-channel fluorescence imaging of surgical areas;

FIG. 3 illustrates a 1D schematic illustration of the filtering process for the presently disclosed system, with 4 filters (A-D) and 4 micro-cameras;

FIG. 4 illustrates an embodiment of the fluorescence multi-camera array microscope using 2 processors to capture and route image data;

FIG. 5 is a schematic illustration of an embodiment of the fluorescence multi-camera array microscope hardware;

FIG. 6 illustrates the fluorescence excitation and emission of 4 fluorophores of interest by the FMCA, along with 3 embodiments of emission filtering layouts for the FMCA;

FIG. 7 illustrates a schematic illustration of 6 different embodiments of emission filtering layouts for the FMCA;

FIG. 8 illustrates a schematic illustration of 2 illumination methods for the FMCA;

FIG. 9 illustrates an embodiment of 2 different illumination methods for the FMCA;

FIG. 10 illustrates workflows for FMCA data post-processing and display;

FIG. 11 illustrates a schematic illustration of the FMCA attached to a robotically controlled arm for dynamic positioning near a surgical area for 3D imaging;

FIG. 12 illustrates two schematic illustrations for alternative illumination methods that the FMCA can be outfitted with;

FIG. 13 illustrates the fluorescence excitation and emission curves of 4 fluorophores of interest by the FMCA, along with the selected excitation and emission passbands for an example embodiment of the FMCA;

FIG. 14 illustrates the FMCA outfitted with external excitation LEDs for video-rate acquisition of 4 fluorescence channels;

FIG. 15 illustrates example visualizations of FMCA data from 16 uniquely filtered micro-cameras; and

FIG. 16 illustrates example 3D visualizations of FMCA data with 4-color fluorescence overlay.

### DETAILED DESCRIPTION OF THE INVENTION

The example embodiments herein and the various features and advantageous details thereof are explained more fully with reference to the non-limiting embodiments that are illustrated in the accompanying drawings and detailed in the following description. Descriptions of well-known components and processing techniques are omitted to not unnecessarily obscure the embodiments herein. The description herein is intended merely to facilitate an understanding of ways in which the example embodiments herein can be practiced and to further enable those of skill in the art to practice the example embodiments herein. Accordingly, this disclosure should not be construed as limiting the scope of the example embodiments herein.

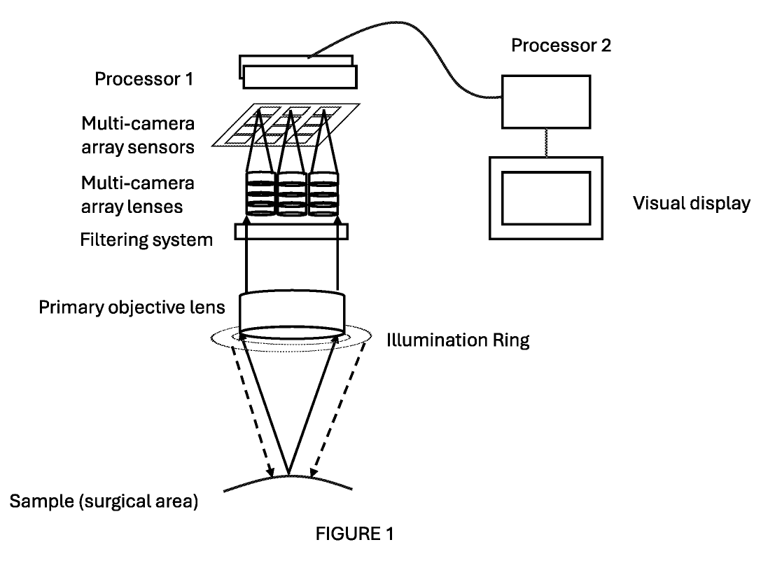

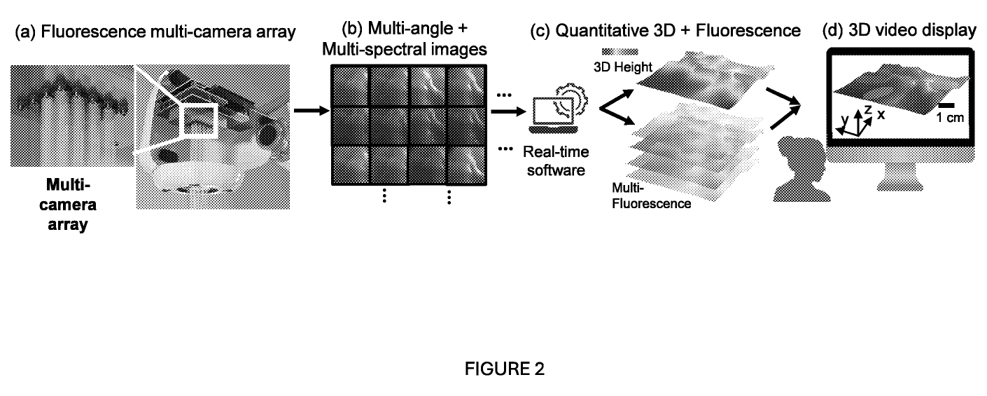

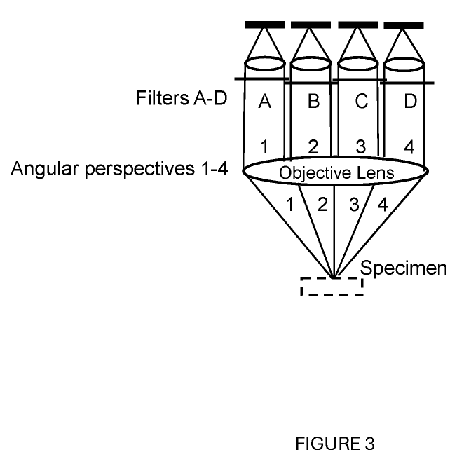

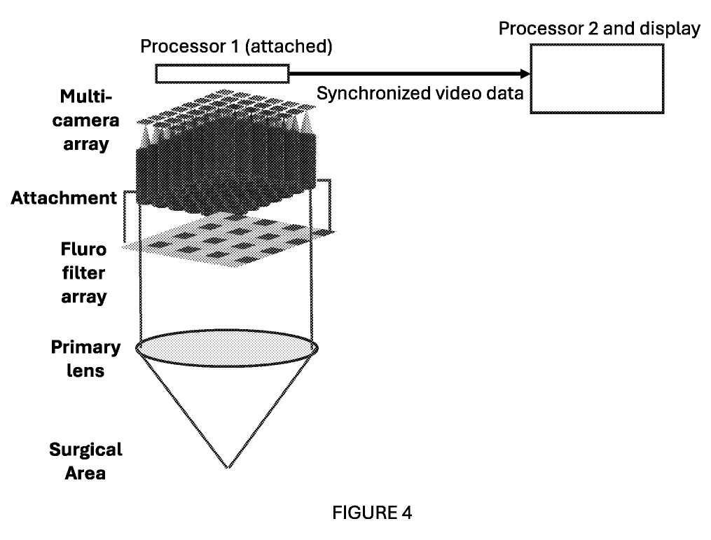

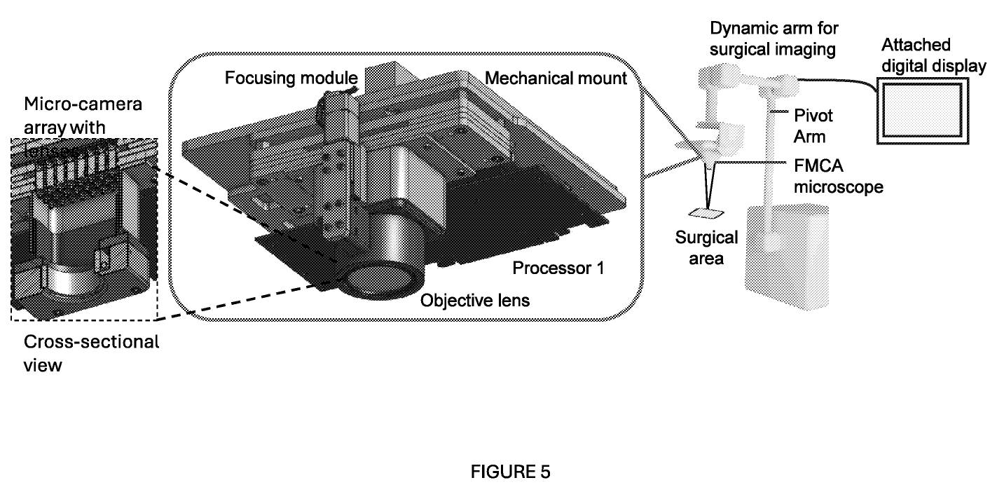

As described more fully below, the present disclosure is directed to a system and method, termed a fluorescence multi-camera array (FMCA), to achieve multi-channel fluorescence imaging of more than two channels, with the ability to measure quantitative 3D information, in real time at high resolution (4K resolution or above) for visual observation during surgical settings. An FMCA exoscope is provided, comprising several key components as outlined below. When integrated together, they offer novel 3D multi-channel fluorescence functionalities (See., e.g., example FMCA exoscope configurations in FIGS. 1-5). Specifically, FIG. 1 shows a schematic illustration of an FMCA microscope imaging a surgical area and displaying the resulting data on a nearby display. FIG. 2 shows a schematic illustration of the system arranged to acquire multiple angular perspectives and multiple filtered fluorescence channels simultaneously, for Quantitative 3D imaging, and multi-channel fluorescence imaging of surgical areas. FIG. 3 shows a 1D schematic illustration of the filtering process for the presently disclosed system, with 4 filters (A-D) and 4 micro-cameras. FIG. 4 shows an embodiment of an FMCA microscope using 2 processors to capture and route image data, and FIG. 5 is a schematic illustration of FMCA microscope hardware.

### Imaging System

In one aspect, an FMCA exoscope is provided that uses a digital imaging system to capture videos for real-time display. The videos can be of ultraviolet, visible, and/or infrared radiation. The digital imaging system contains more than two digital image sensors, which can each be a CMOS sensor, charge coupled device sensor, single-photon avalanche diode (SPAD) array sensor, any format of near-infrared, infrared or thermal image sensor or focal plane array. Different combinations of different formats of digital image sensor can comprise the more than two imaging sensors. In addition, the image sensors can include filters over their pixels to enable multi-spectral and/or polarimetric image capture. In one preferred embodiment, a subset of the two or more digital image sensors include red, green and blue (RGB) color filters to capture colored bright-field image data. In another preferred embodiment, a subset of the two or more digital image sensors include polarimetric filters (e.g., as from current digital CMOS sensors for polarimetric imaging made by Sony). In one preferred embodiment, the array of image sensors is comprised of more than two identical CMOS image sensors. These more than two image sensors are packed together in a tight array to fit behind a primary objective lens. In one preferred embodiment, the more than two image sensors are integrated onto a common printed circuit board with data that is sent to a local FPGA for downstream processing.

Each of the one or more image sensors has its own imaging optic (a single lens or mutli-lens imaging element) placed above it. In one preferred embodiment, these more than two imaging optics are focused towards infinity to capture radiation from a near-parallel set of beams formed by a primary objective lens. In a preferred embodiment, one or more of the imaging optics are connected to a focusing module that can display the imaging optic with respect to its associated image sensor. Alternatively, one or more of the image sensors may be moved with respect to the associated imaging optic with a secondary focusing module. As used herein, the pair of an image sensor and its associated imaging optic is referred to as a micro-camera.

The above array of more than two micro-cameras is optimized to operate in conjunction with a primary objective lens, which is placed in front of the compact array of micro-cameras to deliver radiation into each micro-camera. The primary objective lens is a multi-element optic that is designed to capture radiation from a sample of interest located nearby. In one preferred embodiment, the sample is located close to one focal length away from the primary objective lens, such that its radiation is approximately collimated into the compact array of micro-cameras. In one preferred embodiment, the primary objective lens is repositioned with respect to the sample and the micro-camera array by a focusing module.

In one preferred embodiment, a tightly packed set of 4 micro-cameras can be used to jointly record video from 4 unique perspectives from the specimen area of interest. In a second preferred embodiment, a tightly packed set of 5 micro-cameras can be used to jointly record video from 5 unique perspectives from the specimen area of interest. In another preferred embodiment, a tightly packed set of 9 micro-cameras can be used to jointly record video from 9 unique perspectives from the specimen area of interest. In another preferred embodiment, a tightly packed set of 12 micro-cameras can be used to jointly record video from 12 unique perspectives from the specimen area of interest. In another preferred embodiment, a tightly packed set of 16 micro-cameras can be used to jointly record video from 16 unique perspectives from the specimen area of interest. The tight packaging can allow the entire imaging system to fit atop a moveable arm for ease of use during surgeries. The minimization of additional electronics and wiring by forming a tight package can likewise lead to simpler systems that are less prone to error and/or failure cases during motion and re-positioning in particular.

In one preferred embodiment, the above imaging system can be operated to capture and process multi-perspective imaging data to render 3D images.

### Filtering System

In a preferred embodiment, radiation filters can be included within the above imaging system to spectrally and/or polarimetrically filter incident radiation. In one preferred embodiment, different spectral filters are included over different groups of the two or more imaging micro-cameras to capture different bands of fluorescence emission. In another preferred embodiment, more than one of the same type of spectral filter is used to filter more than one micro-camera, while more than one micro-camera within the system does not include a spectral filter. The filters can be arranged in unique patterns, such as interleaved checkerboard patterns or custom-selected patterns to optimize the signal-to-noise ratio and 3D image rendering accuracy of resulting processing. In one preferred embodiment, the bright-field cameras (for RGB imaging, without spectral filters) are arranged towards the periphery of the array, while the spectrally filtered micro-cameras are arranged towards the center of the array.

While a standard implementation of a micro-camera array includes micro-cameras within a rectilinear grid, it is also possible to implement the presently disclosed invention with a non-rectilinear packing of micro-cameras. A non-rectilinear packing may provide a better packing density of micro-cameras (which typically have cylindrical lenses) behind the optically addressed area of the primary objective lens, which typically covers a circular area.

In an example embodiment, 16 micro-cameras comprise the imaging system array. 4 of the micro-cameras capture RGB color imagery and video without a spectral filter. 3 of the micro-cameras include spectral filter type A, which exhibits a particular spectral pass-band curve. 3 of the micro-cameras include spectral filter type B, which exhibits a different spectral pass-band curve. 3 of the micro-cameras include spectral filter type C, which exhibits a different spectral pass-band curve. 3 of the micro-cameras include spectral filter type D, which exhibits a different spectral pass-band curve. In total, 4 different spectral pass-bands are captured by the system for to resolve a number of unique fluorescence spectra of interest, while a subset of 4 micro-cameras are also available to capture bright-field imagery.

In an example embodiment, 12 micro-cameras comprise the imaging system array. 4 of the micro-cameras capture RGB color imagery and video without a spectral filter. 2 of the micro-cameras include spectral filter type A, which exhibits a particular spectral pass-band curve. 2 of the micro-cameras include spectral filter type B, which exhibits a different spectral pass-band curve. 2 of the micro-cameras include spectral filter type C, which exhibits a different spectral pass-band curve. 2 of the micro-cameras include spectral filter type D, which exhibits a different spectral pass-band curve. In total, 4 different spectral pass-bands are captured by the system for to resolve a number of unique fluorescence spectra of interest, while a subset of 4 micro-cameras are also available to capture bright-field imagery.

In an example embodiment, 12 micro-cameras comprise the imaging system array. 4 of the micro-cameras capture RGB color imagery and video without a spectral filter. 2 of the micro-cameras include spectral filter type A, which exhibits a particular spectral pass-band curve. 2 of the micro-cameras include spectral filter type B, which exhibits a different spectral pass-band curve. 2 of the micro-cameras include spectral filter type C, which exhibits a different spectral pass-band curve. 2 of the micro-cameras include spectral filter type D, which exhibits a different spectral pass-band curve. In total, 4 different spectral pass-bands are captured by the system for to resolve a number of unique fluorescence spectra of interest, while a subset of 4 micro-cameras are also available to capture bright-field imagery.

In an example embodiment, 12 micro-cameras comprise the imaging system array. 3 of the micro-cameras capture RGB color imagery and video without a spectral filter. 3 of the micro-cameras include spectral filter type A, which exhibits a particular spectral pass-band curve. 3 of the micro-cameras include spectral filter type B, which exhibits a different spectral pass-band curve. 3 of the micro-cameras include spectral filter type C, which exhibits a different spectral pass-band curve. In total, 3 different spectral pass-bands are captured by the system for to resolve a number of unique fluorescence spectra of interest, while a subset of 3 micro-cameras are also available to capture bright-field imagery.

In an example embodiment, 9 micro-cameras comprise the imaging system array. 3 of the micro-cameras capture RGB color imagery and video without a spectral filter. 2 of the micro-cameras include spectral filter type A, which exhibits a particular spectral pass-band curve. 2 of the micro-cameras include spectral filter type B, which exhibits a different spectral pass-band curve. 2 of the micro-cameras include spectral filter type C, which exhibits a different spectral pass-band curve. In total, 3 different spectral pass-bands are captured by the system for to resolve a number of unique fluorescence spectra of interest, while a subset of 3 micro-cameras are also available to capture bright-field imagery.

In an example embodiment, 9 micro-cameras comprise the imaging system array. 3 of the micro-cameras capture RGB color imagery and video without a spectral filter. 3 of the micro-cameras include spectral filter type A, which exhibits a particular spectral pass-band curve. 3 of the micro-cameras include spectral filter type B, which exhibits a different spectral pass-band curve. In total, 2 different spectral pass-bands are captured by the system for to resolve a number of unique fluorescence spectra of interest, while a subset of 3 micro-cameras are also available to capture bright-field imagery.

In an example embodiment, 9 micro-cameras comprise the imaging system array. 5 of the micro-cameras capture RGB color imagery and video without a spectral filter. 1 of the micro-cameras include spectral filter type A, which exhibits a particular spectral pass-band curve. 1 of the micro-cameras include spectral filter type B, which exhibits a different spectral pass-band curve. 1 of the micro-cameras include spectral filter type C, which exhibits a different spectral pass-band curve. 1 of the micro-cameras include spectral filter type D, which exhibits a different spectral pass-band curve. In total, 4 different spectral pass-bands are captured by the system for to resolve a number of unique fluorescence spectra of interest, while a subset of 5 micro-cameras are also available to capture bright-field imagery.

In an example embodiment, 8 micro-cameras comprise the imaging system array. 2 of the micro-cameras capture RGB color imagery and video without a spectral filter. 2 of the micro-cameras include spectral filter type A, which exhibits a particular spectral pass-band curve. 2 of the micro-cameras include spectral filter type B, which exhibits a different spectral pass-band curve. 2 of the micro-cameras include spectral filter type C, which exhibits a different spectral pass-band curve. In total, 3 different spectral pass-bands are captured by the system for to resolve a number of unique fluorescence spectra of interest, while a subset of 2 micro-cameras are also available to capture bright-field imagery.

In an example embodiment, 7 micro-cameras comprise the imaging system array. 4 of the micro-cameras capture RGB color imagery and video without a spectral filter. 1 of the micro-cameras include spectral filter type A, which exhibits a particular spectral pass-band curve. 1 of the micro-cameras include spectral filter type B, which exhibits a different spectral pass-band curve. 1 of the micro-cameras include spectral filter type C, which exhibits a different spectral pass-band curve. In total, 3 different spectral pass-bands are captured by the system for to resolve a number of unique fluorescence spectra of interest, while a subset of 4 micro-cameras are also available to capture bright-field imagery.

In an example embodiment, 7 micro-cameras comprise the imaging system array. 3 of the micro-cameras capture RGB color imagery and video without a spectral filter. 1 of the micro-cameras include spectral filter type A, which exhibits a particular spectral pass-band curve. 1 of the micro-cameras include spectral filter type B, which exhibits a different spectral pass-band curve. 1 of the micro-cameras include spectral filter type C, which exhibits a different spectral pass-band curve. 1 of the micro-cameras include spectral filter type D, which exhibits a different spectral pass-band curve. In total, 4 different spectral pass-bands are captured by the system for to resolve a number of unique fluorescence spectra of interest, while a subset of 3 micro-cameras are also available to capture bright-field imagery.

In an example embodiment, 6 micro-cameras comprise the imaging system array. 2 of the micro-cameras capture RGB color imagery and video without a spectral filter. 1 of the micro-cameras include spectral filter type A, which exhibits a particular spectral pass-band curve. 1 of the micro-cameras include spectral filter type B, which exhibits a different spectral pass-band curve. 1 of the micro-cameras include spectral filter type C, which exhibits a different spectral pass-band curve. 1 of the micro-cameras include spectral filter type D, which exhibits a different spectral pass-band curve. In total, 4 different spectral pass-bands are captured by the system for to resolve a number of unique fluorescence spectra of interest, while a subset of 2 micro-cameras are also available to capture bright-field imagery.

In an example embodiment, 6 micro-cameras comprise the imaging system array. 3 of the micro-cameras capture RGB color imagery and video without a spectral filter. 1 of the micro-cameras include spectral filter type A, which exhibits a particular spectral pass-band curve. 1 of the micro-cameras include spectral filter type B, which exhibits a different spectral pass-band curve. 1 of the micro-cameras include spectral filter type C, which exhibits a different spectral pass-band curve. In total, 3 different spectral pass-bands are captured by the system for to resolve a number of unique fluorescence spectra of interest, while a subset of 3 micro-cameras are also available to capture bright-field imagery.

In an example embodiment, 5 micro-cameras comprise the imaging system array. 3 of the micro-cameras capture RGB color imagery and video without a spectral filter. 1 of the micro-cameras include spectral filter type A, which exhibits a particular spectral pass-band curve. 1 of the micro-cameras include spectral filter type B, which exhibits a different spectral pass-band curve. In total, 2 different spectral pass-bands are captured by the system for to resolve a number of unique fluorescence spectra of interest, while a subset of 3 micro-cameras are also available to capture bright-field imagery.

In an example embodiment, 5 micro-cameras comprise the imaging system array. 2 of the micro-cameras capture RGB color imagery and video without a spectral filter. 1 of the micro-cameras include spectral filter type A, which exhibits a particular spectral pass-band curve. 1 of the micro-cameras include spectral filter type B, which exhibits a different spectral pass-band curve. 1 of the micro-cameras include spectral filter type C, which exhibits a different spectral pass-band curve. In total, 3 different spectral pass-bands are captured by the system for to resolve a number of unique fluorescence spectra of interest, while a subset of 2 micro-cameras are also available to capture bright-field imagery.

In any of the above embodiments, the primary objective lens may also include a filter for additional spectral and/or polarimetric filtering. This filter will alter the incoming radiation for example to enhance blockage of fluorescence excitation radiation, filter out unwanted spectral bands, to improve overall contrast with polarization filtering, and/or to control total brightness with neutral density filtering.

### Illumination System

For effective simultaneous recording of bright-field and multi-channel fluorescence video, the presently disclosed system also includes a unique illumination platform. In one preferred embodiment, an external light may be used for both bright-field and excitation. This external light may take the form of a ring LED array, which includes more than one LED packaged closely together, which is positioned to surround the primary objective lens. The external ring LED array includes more than one type of LED, each associated with one of the channels that is detecting radiation. Different excitation filters are additionally included to cover specific sets of LEDs to improve the signal-to-noise ratio, background and overall quality of detected multi-channel fluorescence imagery and video. The power of different sets of LEDs is individually adjustable to maximize the quality of multi-channel fluorescence excitation and bright-field imaging. In one preferred embodiment, the ring LED array includes a set of 5 different groups of LED, where each group consists of more than one LED. One group of the set of 5 groups consists of white LEDs optimized for bright-field imaging, a second group consists of LEDs and associated excitation filters optimized to detect fluorescence channel A, a third group consists of LEDs and associated excitation filters optimized to detect fluorescence channel B, a forth group consists of LEDs and associated excitation filters optimized to detect fluorescence channel C, and a fifth group consists of LEDs and associated excitation filters optimized to detect fluorescence channel D. In another preferred embodiment, the ring LED array includes a set of 4 different groups of LED, where each group consists of more than one LED. One group of the set of 4 groups consists of white LEDs optimized for bright-field imaging, a second group consists of LEDs and associated excitation filters optimized to detect fluorescence channel A, a third group consists of LEDs and associated excitation filters optimized to detect fluorescence channel B, and a forth group consists of LEDs and associated excitation filters optimized to detect fluorescence channel C. In one preferred embodiment, the ring LED array includes a set of 3 different groups of LED, where each group consists of more than one LED. One group of the set of 3 groups consists of white LEDs optimized for bright-field imaging, a second group consists of LEDs and associated excitation filters optimized to detect fluorescence channel A, and a third group consists of LEDs and associated excitation filters optimized to detect fluorescence channel B.

In another preferred embodiment, specimen illumination and multi-channel fluorescence excitation can come from an array of LEDs that are internal to the FMCA unit. This internal array of LEDs includes clear apertures that allow emission light to pass through to each micro-camera for subsequent imaging. It can fit atop and be mechanically attached the micro-camera array. The internal LED array includes more than one type of LED, each associated with one of the channels that is detecting radiation. Different excitation filters are additionally included to cover specific sets of LEDs to improve the signal-to-noise ratio, background and overall quality of detected multi-channel fluorescence imagery and video. The power of different sets of LEDs is individually adjustable to maximize the quality of multi-channel fluorescence excitation and bright-field imaging. In one preferred embodiment, the internal LED array includes a set of 5 different groups of LED, where each group consists of more than one LED. One group of the set of5groups consists of white LEDs optimized for bright-field imaging, a second group consists of LEDs and associated excitation filters optimized to detect fluorescence channel A, a third group consists of LEDs and associated excitation filters optimized to detect fluorescence channel B, a forth group consists of LEDs and associated excitation filters optimized to detect fluorescence channel C, and a fifth group consists of LEDs and associated excitation filters optimized to detect fluorescence channel D. In another preferred embodiment, the internal LED array includes a set of 4 different groups of LED, where each group consists of more than one LED. One group of the set of 4 groups consists of white LEDs optimized for bright-field imaging, a second group consists of LEDs and associated excitation filters optimized to detect fluorescence channel A, a third group consists of LEDs and associated excitation filters optimized to detect fluorescence channel B, and a forth group consists of LEDs and associated excitation filters optimized to detect fluorescence channel C. In one preferred embodiment, the internal LED array includes a set of 3 different groups of LED, where each group consists of more than one LED. One group of the set of 3 groups consists of white LEDs optimized for bright-field imaging, a second group consists of LEDs and associated excitation filters optimized to detect fluorescence channel A, and a third group consists of LEDs and associated excitation filters optimized to detect fluorescence channel B.

In another preferred embodiment, both an external LED ring array and an internal LED array may be used for specimen illumination and multi-channel fluorescence excitation. Both array sources can be turned on simultaneously, and the total power of each array may be adjusted between 0% and 100% to optimize and maximize the quality of multi-channel fluorescence excitation and bright-field imaging.

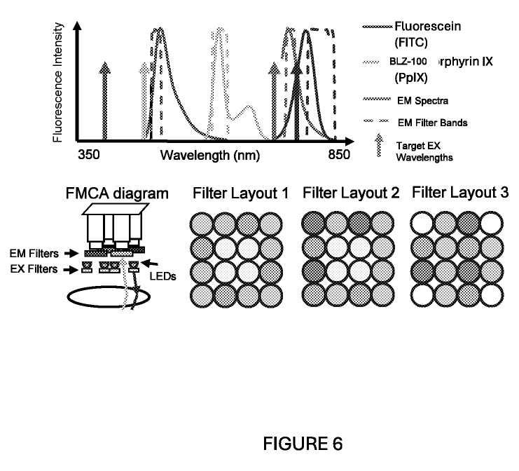

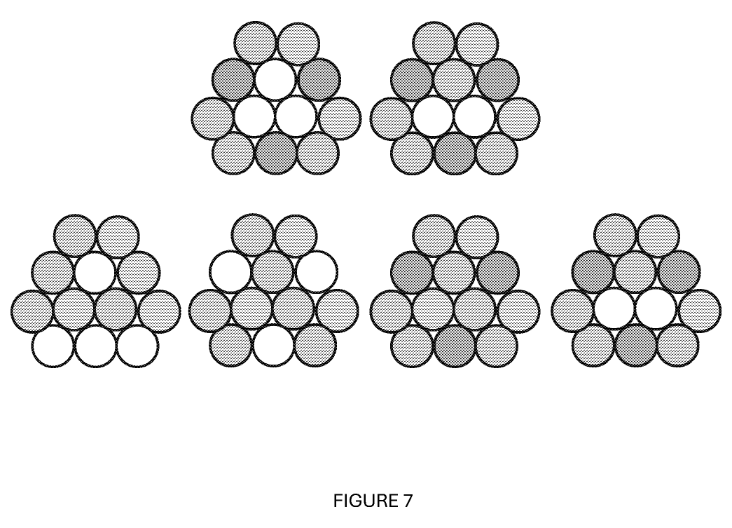

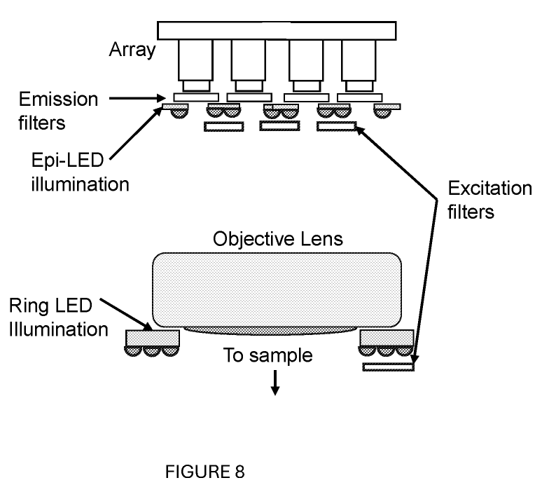

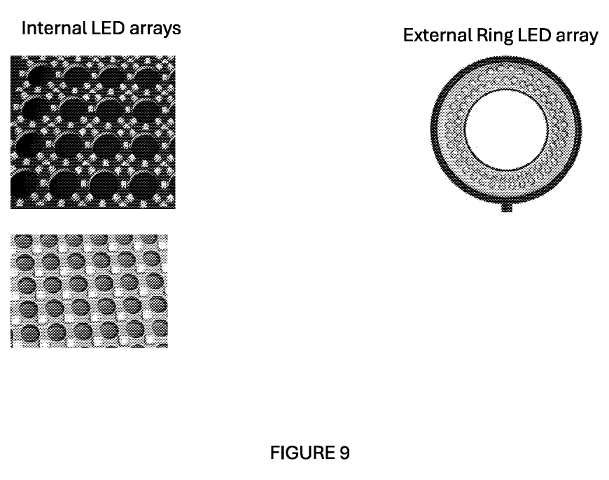

The filtering array arrangement of emission filters and the LED illumination arrangement with excitation filters is carefully designed to achieve simultaneous multi-channel excitation for simultaneous fluorescence emission imaging. An example of simultaneously exciting and detecting 4 fluorophores that are commonly used in surgical imaging is shown in FIG. 6. Here, several unique emission filtering arrangements are shown for an array of 16 micro-cameras, where each colored circle represents a filter type. A similar set of 6 unique emission filter arrangements for use with a 12 micro-camera array of non-rectangular packing is shown in FIG. 7. FIG. 8 shows the joint use of an internal LED array and an external ring LED array for specimen illumination and excitation. FIG. 9 shows example LED arrays.

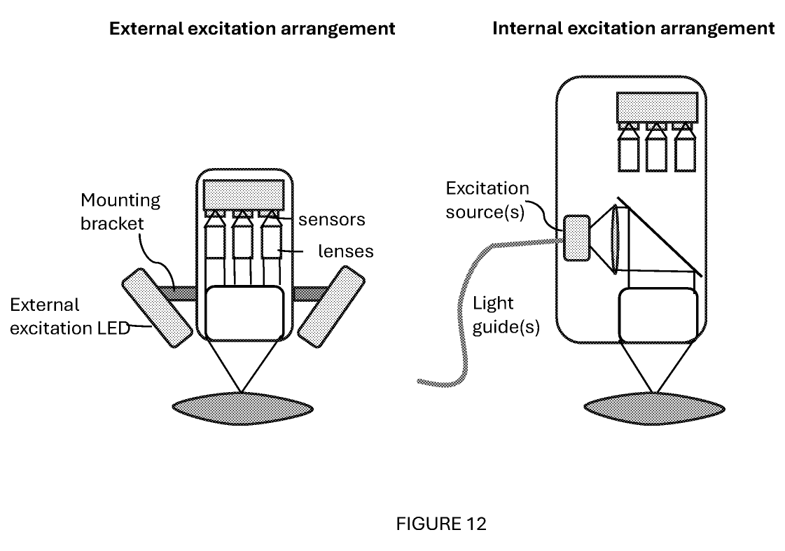

In other embodiments, FIG. 12 illustrates two schematic illustrations for alternative illumination methods that the FMCA can be outfitted with.

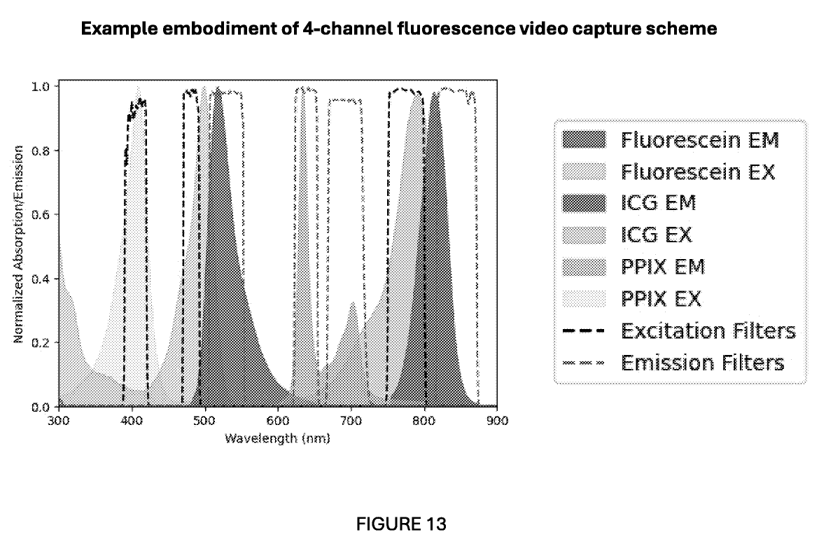

In further embodiments, FIG. 13 illustrates the fluorescence excitation and emission curves of 4 fluorophores of interest by the FMCA, along with the selected excitation and emission passbands for an example embodiment of the FMCA.

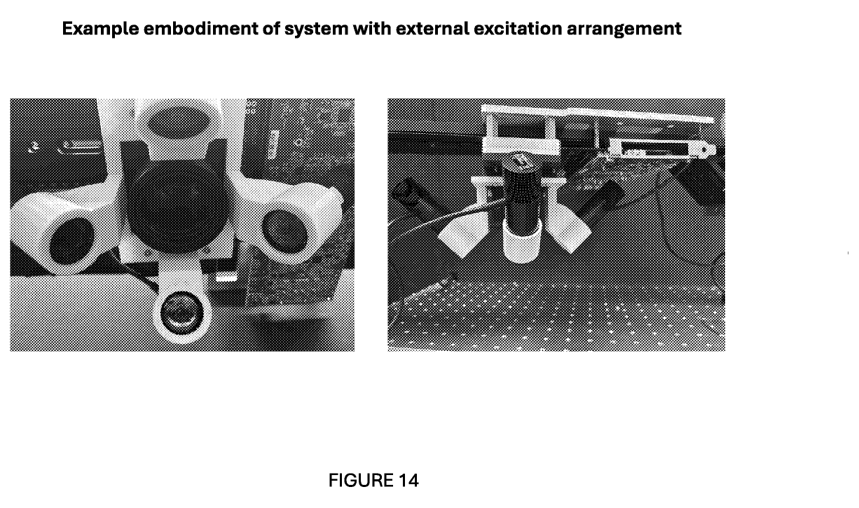

In other embodiments, FIG. 14 illustrates the FMCA outfitted with external excitation LEDs for video-rate acquisition of 4 fluorescence channels.

### Data Management System

The present invention includes a data management system to control and process incoming video streams of data to enhance surgical workflows. The more than two imaging systems outlined above are configured to capture synchronized image or video data. The included data management system is designed to control the synchronous capture, exposure time, gamma, balance, min and max exposure, and other imaging properties of the image sensors. In one preferred embodiment, a first processor is used to perform the above control functionalities. The included data management system is also designed to route synchronous image or video data from the two or more image sensors to this first processor. The first processor can take the form of one or more FPGAs, one or more CPUs, one or more GPUs, or one or more compact computational modules that fit within the FMCA imaging system package.

In one preferred embodiment of the data management system, a second processor is connected to the first processor to form a complete data management system for the FMCA microscope. The second processor can take the form of a computer, one or more FPGAs, one or more CPUs, one or more GPUs, or one or more compact computational modules. The second processor additionally processes incoming video data to render 3D images, to register associated micro-camera images for subsequent real-time display, to digitally refocus captured image data (as it resembles a light field, and is amenable to digital refocusing), crops incoming image data, or otherwise processes the data to track, locate, segment or highlight particular objects, features or image areas.

The system can be used with pulsed light sources illuminating the body to capture pulsed fluorescence image data, which can include fluorescence lifetime signals and other fluorescent data that minimizes background autofluorescence.

### Visual Display and Interface

The video data from the FMCA microscope is routed to a second processor that both post-processes the incoming video streams, displays the rendered results on a display for real-time visualization, and sends associated image and video-derived calculations to peripheral devices that can include motion control systems, focusing mechanisms, robotic manipulation systems such as micro-injectors, patch clamp systems, tissue ablation tools, electrophysiology equipment and other specimen manipulation tools that are connected to micro-mechanical motion controllers.

In one preferred embodiment, 3D information about the specimen area is derived from the FMCA’s multiple micro-camera video streams. This 3D information takes the form of 3D coordinates at one or more spatial location across the imaging field-of-view, which then can be displayed on the visual interface (each as a number or a color to denote height or a rendered surface or volume), and additionally used to drive and update attached peripheral devices such as those listed above.

In addition, unique multi-spectral information about the specimen area is derived from the FMCA’s multiple micro-camera video streams. For fluorescence specimens, this multi-spectral information takes the form of unique spectral measurements at given spatial locations across the imaging field of view, which then can be displayed on the visual interface (each as a number or a color to denote the brightness of the associated spectral channel) and additionally used to drive and update attached peripheral devices such as those listed above. The multi-spectral information includes additional specificity about the sample area of interest that can feed post-processing algorithms such as object detection, region detection, segmentation, or other more advanced spatial localization algorithms to identify features of interest to highlight on the visual display and to enhance the visual properties of the display and/or to improve the control of attached peripheral devices.

In one preferred embodiment, bright-field video data is input into an algorithm that derives 3D information about the specimen area. Fluorescence data is input into a second algorithm that derives 3D information about the specimen area. The two resulting 3D derivations are merged to then jointly display video of bright-field imagery of the specimen area along with fluorescence video of the specimen area in an interface with a 3D relief map. Users have the ability to toggle on and off the merger and overlay of each channel of the captured fluorescence image data atop the bright-field image data.

In another preferred embodiment, bright-field video data is input into an algorithm that derives 3D information about the specimen area. Fluorescence data is input into a second algorithm that derives 3D information about the specimen area. The two resulting 3D derivations are merged to then jointly display video of bright-field imagery of the specimen area along with fluorescence video of the specimen area in an interface with quantitative values denoting the depth or height of one or more features within the field-of-view. These features can include fluorescence areas on the sample, surgical tools or other external devices that are visible within the field of view, or other tissue areas of interest within the field-of-view, for example.

In addition to the visual display interface, additional downstream processing algorithms may be applied to the captured data, including video compression, preparation of FMCA video data for remote viewing via a web browser, and merger of multiple FMCA video streams.

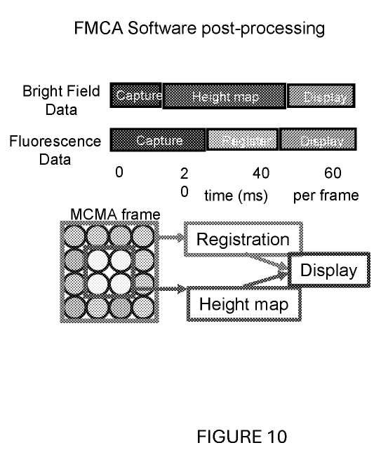

In other embodiments, FIG. 10 shows an example configuration for rapid data processing for real-time multi-channel 3D video display, and FIG. 14 illustrates the FMCA outfitted with external excitation LEDs for video-rate acquisition of 4 fluorescence channels.

### Benefits Towards Compact Surgical Microscope Design

The innovative components described herein allow the presently disclosed system to integrate within a novel microscope design that uses a compact array of lenses and sensors to acquire multi-perspective image data. In one preferred embodiment, the above-described sensor array and electronics package may be integrated into a moveable microscope system that is integrated on a mechanical arm. Such systems are commonly used within surgical scenarios, e.g. to perform microsurgery, ophthalmology, in neurosurgery, dermatology and other clinical applications. Here, many image sensors are required to be packed together closely to fit within the required optical footprint of typical macro-objective lenses used within microsurgery setups. The image sensors, which in one preferred embodiment each have an associated lens system in front to redirect light from a portion of the macro-objective lens, are placed in a tight array behind the macro-objective lens. The resulting setup can lead to the formation of high-resolution images onto each image sensor, where each image is captured from a unique angular perspective. This angular perspective data can be used for stereo-image viewing or to compute 3D information from the acquired specimen area of interest. In addition, it can be uniquely filtered to jointly detect fluorescence emission from surgical area surfaces. Associated imagery is then fused for real-time 3D display on a head’s up monitor. It can additionally be processed for 2D tracking, 3D tracking, 2D segmentation, 3D segmentation, and other associated analysis overlays during visualization. This quantitative 3D data can also drive robotically controlled instrumentation. The capture of multi-channel fluorescence data similarly offers more information about the surgical surface that can be of molecular, biochemical, genetic, transcriptomic, proteomic, multi-ohmic or other functional specificity, which further enhances existing surgical workflows (automated workflows, semi-automated workflows or manual workflows).

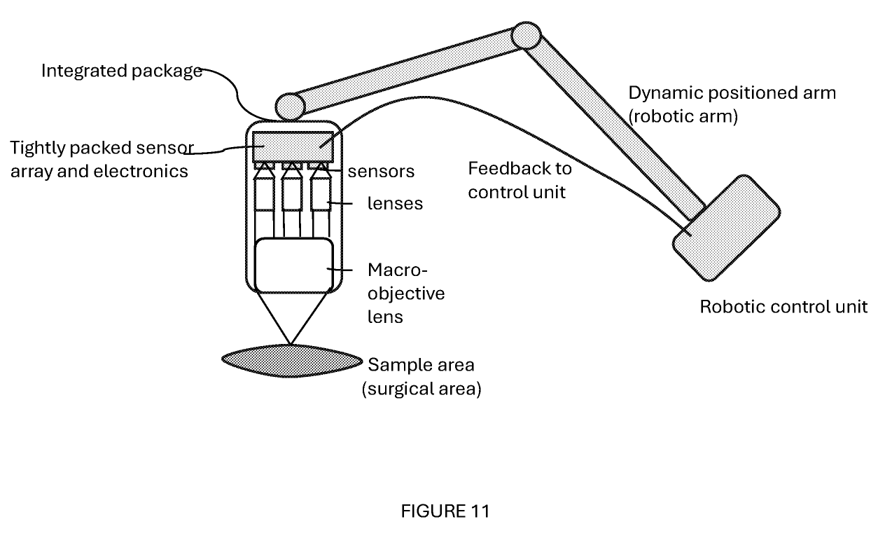

In other embodiments, FIG. 11 illustrates a schematic illustration of the FMCA attached to a robotically controlled arm for dynamic positioning near a surgical area for 3D imaging.

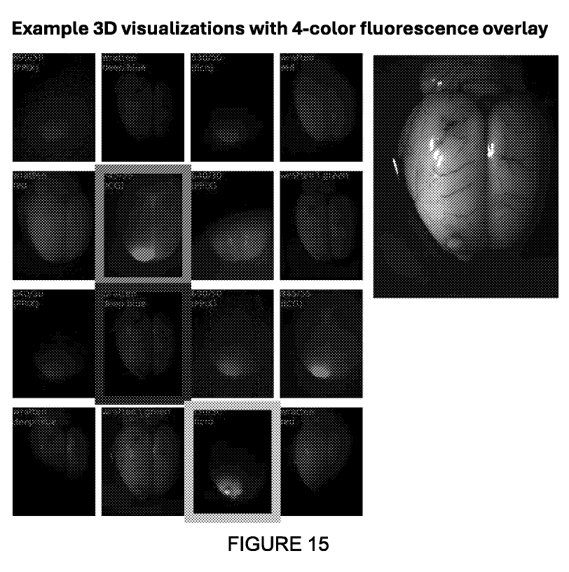

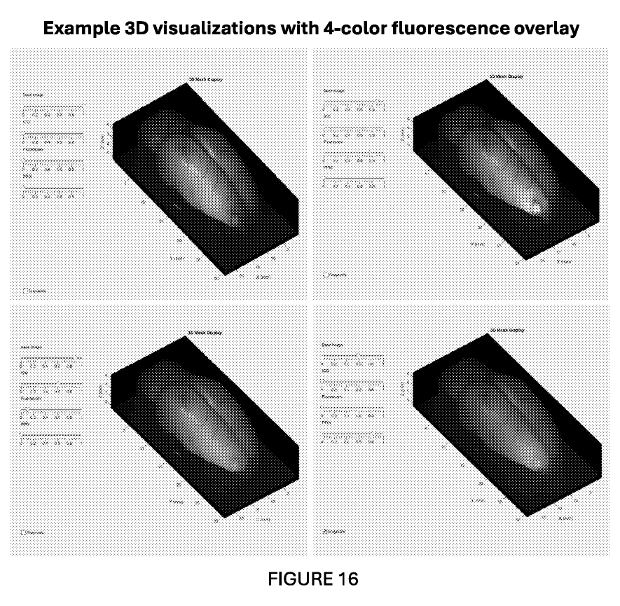

In other embodiments, FIG. 15 illustrates example visualizations of FMCA data from 16 uniquely filtered micro-cameras, and FIG. 16 illustrates example 3D visualizations of FMCA data with 4-color fluorescence overlay.

### General Definitions

It will be appreciated that various aspects of the disclosure may be embodied as a method, system, computer readable medium, and/or computer program product. Aspects of the disclosure may take the form of hardware embodiments, software embodiments (including firmware, resident software, micro-code, etc.), or embodiments combining software and hardware aspects that may all generally be referred to herein as a “circuit,” “module,” or “system.” Furthermore, the methods of the disclosure may take the form of a computer program product on a computer-usable storage medium having computer-usable program code embodied in the medium.

Any suitable computer useable medium may be utilized for software aspects of the disclosure. The computer-usable or computer-readable medium may be, for example but not limited to, an electronic, magnetic, optical, electromagnetic, infrared, or semiconductor system, apparatus, device, or propagation medium. The computer readable medium may include transitory and/or non-transitory embodiments. More specific embodiments (a non-exhaustive list) of the computer-readable medium would include some or all of the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission medium such as those supporting the Internet or an intranet, or a magnetic storage device. Note that the computer-usable or computer-readable medium may even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device.

Program code for carrying out operations of the disclosure may be written in an object-oriented programming language such as Java, Smalltalk, C++ or the like. However, the program code for carrying out operations of the disclosure may also be written in conventional procedural programming languages, such as the “C” programming language or similar programming languages. The program code may be executed by a processor, application specific integrated circuit (ASIC), or other component that executes the program code. The program code may be simply referred to as a software application that is stored in memory (such as the computer readable medium discussed above). The program code may cause the processor (or any processor-controlled device) to produce a graphical user interface (“GUI”). The graphical user interface may be visually produced on a display device, yet the graphical user interface may also have audible features. The program code, however, may operate in any processor-controlled device, such as a computer, server, personal digital assistant, phone, television, or any processor-controlled device utilizing the processor and/or a digital signal processor.

The program code may locally and/or remotely execute. The program code, for example, may be entirely or partially stored in local memory of the processor-controlled device. The program code, however, may also be at least partially remotely stored, accessed, and downloaded to the processor-controlled device. A user’s computer, for example, may entirely execute the program code or only partly execute the program code. The program code may be a stand-alone software package that is at least partly on the user’s computer and/or partly executed on a remote computer or entirely on a remote computer or server. In the latter scenario, the remote computer may be connected to the user’s computer through a communications network.

The disclosure may be applied regardless of networking environment. The communications network may be a cable network operating in the radio-frequency domain and/or the Internet Protocol (IP) domain. The communications network, however, may also include a distributed computing network, such as the Internet (sometimes alternatively known as the “World Wide Web”), an intranet, a local-area network (LAN), and/or a wide-area network (WAN). The communications network may include coaxial cables, copper wires, fiber optic lines, and/or hybrid-coaxial lines. The communications network includes wireless portions utilizing any portion of the electromagnetic spectrum and any signaling standard (such as the IEEE 802 family of standards, GSM/CDMA/TDMA or any cellular standard, and/or the ISM band). The communications network may even include powerline portions, in which signals are communicated via electrical wiring. The disclosure may be applied to any wireless/wireline communications network, regardless of physical componentry, physical configuration, or communications standard(s).

In some aspects, wireless communication interfaces may include, but are not limited to, an Intranet connection, Internet, Personal Area Networks (PANs) for the exchange of data over short distances, e.g., using short-wavelength radio transmissions in the industrial, scientific, and medical (ISM) band ISM band from 2400-2480 MHz) from fixed and mobile devices (e.g., Bluetooth® technology), wireless fidelity (Wi-Fi), Wi-Max, IEEE 802.1 1 technology, radio frequency (RF), Infrared Data Association (IrDA) compatible protocols, Local Area Networks (LANs), Wide Area Networks (WANs), Shared Wireless Access Protocol (SWAP), Zigbee, Near-Field Communication (NFC), LiFi, 5G, any combinations thereof, and other types of wireless networking protocols.

Certain aspects of disclosure are described with reference to various methods and method steps. It will be understood that each method step can be implemented by the program code and/or by machine instructions. The program code and/or the machine instructions may create means for implementing the functions/acts specified in the methods.

The program code may also be stored in a computer-readable memory that can direct the processor, computer, or other programmable data processing apparatus to function in a particular manner, such that the program code stored in the computer-readable memory produce or transform an article of manufacture including instruction means which implement various aspects of the method steps.

The program code may also be loaded onto a computer or other programmable data processing apparatus to cause a series of operational steps to be performed to produce a processor/computer implemented process such that the program code provides steps for implementing various functions/acts specified in the methods of the disclosure.

Any of a variety of light sources may be used to provide the excitation and/or imaging light, including but not limited to, tungsten lamps, tungsten-halogen lamps, arc lamps, lasers, light emitting diodes (LEDs), or laser diodes.

Terms and phrases used in this document, and variations thereof, unless otherwise expressly stated, should be construed as open ended as opposed to limiting. As examples of the foregoing: the term “including” should be read as mean “including, without limitation” or the like; the term “example” is used to provide exemplary instances of the item in discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although item, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter of the present disclosure. For example, the term “about,” when referring to a value can be meant to encompass variations of, in some embodiments ± 100%, in some embodiments ± 50%, in some embodiments ± 20%, in some embodiments ± 10%, in some embodiments ± 5%, in some embodiments ± 1%, in some embodiments ± 0.5%, and in some embodiments ± 0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of embodiments, those skilled in the art will recognize that the embodiments herein can be practiced with modification within the spirit and scope of the embodiments as described herein.

The foregoing description and accompanying figures illustrate the principles, embodiments and modes of operation of the disclosure. However, the disclosure should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the disclosure as defined by the following claims.

1. A surgical imaging system comprising:
(a) a primary objective lens configured to receive radiation from a surgical field;
(b) a plurality of micro-cameras positioned behind the primary objective lens, each micro-camera comprising an image sensor and an associated imaging optic, wherein the plurality of micro-cameras comprises more than two micro-cameras arranged to capture the surgical field from different angular perspectives;
(i) wherein a first subset of the micro-cameras is configured to capture bright-field image data; and
(ii) wherein a second subset of the micro-cameras comprises different spectral emission filters corresponding to a plurality of fluorescence channels;
(c) an illumination system configured to simultaneously illuminate the surgical field for bright-field imaging and excite a plurality of fluorophores; and
(d) one or more processors configured to receive synchronized image data from the plurality of micro-cameras and to generate quantitative three-dimensional information and multi-channel fluorescence image data in real time.

2. The system of claim 1, wherein the plurality of fluorescence channels comprises at least three fluorescence channels.

3. The system of claim 1, wherein the plurality of fluorescence channels comprises four or more fluorescence channels captured simultaneously.

4. The system of claim 1, wherein the plurality of micro-cameras comprises between five and sixteen micro-cameras.

5. The system of claim 1, wherein the micro-cameras are arranged in a rectilinear
or non-rectilinear array to maximize packing density behind the primary objective lens.

6. The system of claim 1, wherein the image sensors comprise at least one of: a Complementary Metal-Oxide-Semiconductor (CMOS) sensor, a charge-coupled device sensor, a single-photon avalanche diode array sensor, or an infrared or thermal focal plane array sensor.

7. The system of claim 1, wherein the micro-camera array comprises more than two identical Complementary Metal-Oxide-Semiconductor (CMOS) image sensors mounted on a common printed circuit board and coupled to a local Field-Programmable Gate Array (FPGA).

8. The system of claim 1, wherein a first subset of the micro-cameras is configured for bright-field imaging and a second subset of the micro-cameras is configured for filtered fluorescence imaging.

9. The system of claim 8, wherein the first subset includes image sensors having Red-Green-Blue (RGB) pixel filters and the second subset includes micro-cameras having emission filters without RGB pixel filters.

10. The system of claim 1, wherein the filtering system comprises at least three different spectral emission filters corresponding to at least three fluorescence channels.

11. The system of claim 1, wherein the illumination system comprises a ring-shaped external light source surrounding the primary objective lens.

12. The system of claim 1, wherein the illumination system further comprises an internal light source positioned between the primary objective lens and the micro-cameras.

13. The system of claim 1, wherein the illumination system comprises independently
controllable excitation light sources corresponding to each fluorescence channel.

14. The system of claim 1, wherein the one or more processors are configured to compute the quantitative three-dimensional information using multi-view stereo reconstruction.

15. The system of claim 1, wherein the quantitative three-dimensional information is independent of illumination intensity.

16. The system of claim 1, further comprising a display configured to present a real-time visualization including bright-field imagery, fluorescence imagery, and/or a three-dimensional representation of the surgical field.

17. The system of claim 16, wherein the fluorescence imagery is overlaid onto the bright-field imagery with depth-dependent visualization.

18. The system of claim 1, wherein the one or more processors are further configured to output the quantitative three-dimensional information to control a robotic or computer-assisted surgical instrument.

19. The system of claim 1, wherein the system operates at video rates and provides spatial resolution of at least 4K per fluorescence channel.

20. A method of surgical imaging comprising:
(a) illuminating a surgical field with bright-field illumination and excitation light for a plurality of fluorophores;
(b) simultaneously capturing bright-field image data and multi-channel fluorescence image data from the surgical field using a plurality of micro-cameras arranged to view the surgical field from different angular perspectives;
(c) processing the captured image data to compute quantitative three-
dimensional information; and
(d) displaying or outputting, in real time, a representation of the surgical field comprising the quantitative three-dimensional information and the multi-channel fluorescence image data.
